(19) United States
(12) Patent Application Publication
(10) Pub. No.: US 2026/0266682 A1
(43) Pub. Date: Sep. 10, 2026

(54) CORRECTING FOR ATMOSPHERIC LEAKS IN RESIDUAL GAS ANALYZER (RGA) TESTING

(71) Applicant: Raytheon Company, Arlington, VA (US)

(72) Inventor: Quinn D. Risch, Tucson, AZ (US)

(21) Appl. No.: 19/071,507

(22) Filed: Mar. 5, 2025

(51) Int. Cl. G01M 3/32; G01N 33/00

(52) U.S. Cl. CPC G01M 3/3281; G01M 3/3236; G01N 33/007; G01N 33/0073

(57) ABSTRACT

A method includes generating a known leak signature based on a leak signal received at a residual gas analyzer (RGA). The method also includes generating a chamber signature based on a chamber signal received at the RGA from a test chamber while a test object is inside the test chamber. The method further includes correcting the chamber signature for atmospheric leaks based on the leak signature to generate a test signature. The test signature corresponds to an atmospheric leak-corrected chamber signature for the test object.

### TECHNICAL FIELD

This disclosure relates generally to residual gas analyzer testing of vacuum systems. More specifically, this disclosure relates to correcting for atmospheric leaks in residual gas analyzer (RGA) testing.

### BACKGROUND

Conventional residual gas analyzer (RGA) testing includes placing a test object inside a test chamber, pumping out the test chamber until the pressure reaches a near vacuum, and measuring a leak rate of one or more gases from the chamber (and the test object) using an RGA. However, the test chamber typically cannot be perfectly sealed. Thus, atmospheric leaks into the test chamber introduce components of the atmosphere into the test chamber. When one or more gases being measured by the RGA as leaks from the test object also exist in the surrounding atmosphere, the RGA signal is contaminated and it appears as though these leaks are emanating from the test object itself.

### SUMMARY

This disclosure relates to correcting for atmospheric leaks in residual gas analyzer (RGA) testing.

In a first embodiment, a method may include generating a known leak signature based on a leak signal received at an RGA. The method may also include generating a chamber signature based on a chamber signal received at the RGA from a test chamber while a test object is inside the test chamber. The method may further include correcting the chamber signature for atmospheric leaks based on the leak signature to generate a test signature. The test signature may correspond to an atmospheric leak-corrected chamber signature for the test object.

Any single one or any combination of the following features may be used with the first embodiment. Correcting the chamber signature for atmospheric leaks based on the leak signature to generate the test signature may include subtracting the leak signature from the chamber signature. The leak signal may be generated by an atmospheric leak standard based on a sample of a testing atmosphere. The leak signal may be received from the atmospheric leak standard at the RGA. Generating the leak signature may include generating the leak signature after receiving the leak signal for at least an amount of time corresponding to a stabilization period for the leak signal. The stabilization period for the leak signal may be about one minute. The atmospheric leak standard may include a capillary leak circuit. The testing atmosphere may include a plurality of atmospheric components. The leak signature, the chamber signature, and the test signature may each include at least a subset of the atmospheric components. Correcting the chamber signature for atmospheric leaks based on the leak signature to generate the test signature may include subtracting the leak signature from the chamber signature until at least one of the atmospheric components in the test signature reaches a value of zero. The chamber signal may be received at the RGA. Generating the chamber signature may include generating the chamber signature after receiving the chamber signal for at least an amount of time corresponding to a stabilization period for the chamber signal. The stabilization period for the chamber signal may be about one minute.

In a second embodiment, a method may include receiving a sample of a testing atmosphere at an RGA through a leak signal and generating a known leak signature based on the leak signal. The method may also include receiving a chamber signal at the RGA from a test chamber while a test object is inside the test chamber and generating a chamber signature based on the chamber signal. The method may further include correcting the chamber signature for atmospheric leaks based on the leak signature to generate a test signature. The test signature may correspond to an atmospheric leak-corrected chamber signature for the test object.

Any single one or any combination of the following features may be used with the second embodiment. Correcting the chamber signature for atmospheric leaks based on the leak signature to generate the test signature may include subtracting the leak signature from the chamber signature. The leak signal may be generated based on the sample of the testing atmosphere with an atmospheric leak standard. Receiving the sample of the testing atmosphere at the RGA through the leak signal may include receiving the leak signal at the RGA from the atmospheric leak standard. Generating the leak signature may include generating the leak signature after receiving the leak signal at the RGA from the atmospheric leak standard for at least an amount of time corresponding to a stabilization period for the leak signal. The atmospheric leak standard may include a capillary leak circuit. The testing atmosphere may include a plurality of atmospheric components. The leak signature, the chamber signature, and the test signature may each include at least a subset of the atmospheric components. Correcting the chamber signature for atmospheric leaks based on the leak signature to generate the test signature may include subtracting the leak signature from the chamber signature until at least one of the atmospheric components in the test signature reaches a value of zero. Generating the chamber signature may include generating the chamber signature after receiving the chamber signal at the RGA from the test chamber for at least an amount of time corresponding to a stabilization period for the chamber signal.

In a third embodiment, a system may include a test chamber, an atmospheric leak standard, an RGA, and a testing processor. The test chamber may be configured to generate a chamber signal while a test object is inside the test chamber. The atmospheric leak standard may be configured to receive a sample of a testing atmosphere and to generate a leak signal based on the sample. The RGA may be coupled to the test chamber and the atmospheric leak standard. The RGA may be configured to receive the leak signal, generate a known leak signature based on the leak signal, receive the chamber signal, and generate a chamber signature based on the chamber signal. The testing processor may be communicatively coupled to the RGA. The testing processor may be configured to correct the chamber signature for atmospheric leaks based on the leak signature to generate a test signature. The test signature may correspond to an atmospheric leak-corrected chamber signature for the test object.

Any single one or any combination of the following features may be used with the third embodiment. The testing processor may be configured to correct the chamber signature for atmospheric leaks based on the leak signature to generate the test signature by subtracting the leak signature from the chamber signature. The testing atmosphere may include a plurality of atmospheric components. The leak signature, the chamber signature, and the test signature may each include at least a subset of the atmospheric components. To subtract the leak signature from the chamber signature, the testing processor may be configured to subtract the leak signature from the chamber signature until at least one of the atmospheric components in the test signature reaches a value of zero. The atmospheric leak standard may include a capillary leak circuit.

Other technical features may be readily apparent to one skilled in the art from the following figures, descriptions, and claims.

### BRIEF DESCRIPTION OF THE DRAWINGS

For a more complete understanding of this disclosure, reference is made to the following description, taken in conjunction with the accompanying drawings, in which:

FIG. 1 illustrates an example of a residual gas analyzer (RGA) testing system supporting correction for atmospheric leaks in RGA testing in accordance with this disclosure;

FIG. 2 illustrates an example of details of the RGA testing equipment of FIG. 1 according to this disclosure;

FIGS. 3A through 3C illustrate graphs depicting examples of RGA spectrums generated during RGA testing with atmospheric leak correction according to this disclosure; and

FIG. 4 illustrates an example of a method for correcting for atmospheric leaks in RGA testing according to this disclosure.

### DETAILED DESCRIPTION

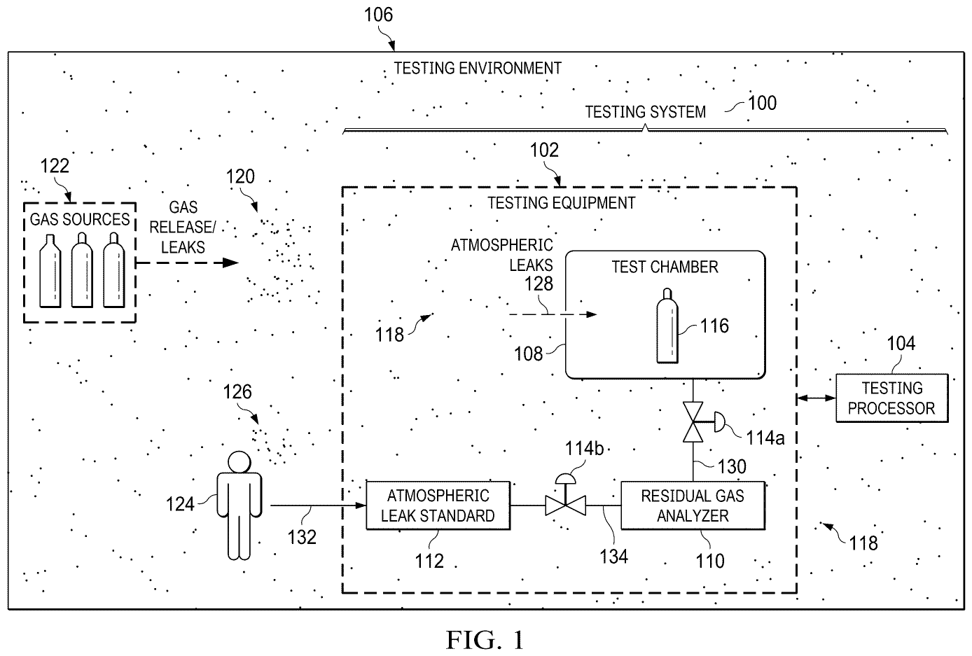

FIGS. 1 through 4, described below, and the various embodiments used to describe the principles of the present disclosure are by way of illustration only and should not be construed in any way to limit the scope of this disclosure. Those skilled in the art will understand that the principles of the present disclosure may be implemented in any type of suitably arranged device or system.

As noted above, conventional residual gas analyzer (RGA) testing includes placing a test object inside a test chamber, pumping out the test chamber until the pressure reaches a near vacuum, and measuring a leak rate of one or more gases from the chamber (and the test object) using an RGA. However, the test chamber typically cannot be perfectly sealed. Thus, atmospheric leaks into the test chamber introduce components of the atmosphere into the test chamber. When one or more gases being measured by the RGA as leaks from the test object also exist in the surrounding atmosphere, the RGA signal is contaminated and it appears as though these leaks are emanating from the test object itself. When the test object does not include any components also found in the atmosphere, removing the contamination can be a simple process. However, when the test object does include any components also found in the atmosphere, it becomes difficult to determine if those components have leaked from the test object or have been introduced through atmospheric leaks.

This disclosure provides a method and system for correcting for atmospheric leaks in RGA testing. As described in more detail below, in some embodiments, a leak signature is generated based on a leak signal received at an RGA. A chamber signature is generated based on a chamber signal received at the RGA from a test chamber while a test object is inside the test chamber. The chamber signature is corrected for atmospheric leaks based on the leak signature to generate a test signature. The test signature corresponds to an atmospheric leak-corrected chamber signature for the test object. As used here, a “signature” refers to a pattern of values corresponding to a leak rate for each of one or more notable components included in a corresponding signal. Thus, the RGA can provide an accurate estimate of atmospheric leaks based on a measurement of the actual atmospheric components provided through the leak signal to allow the RGA testing system to account for atmospheric leaks. Those estimated atmospheric leaks can be removed from the chamber signature in generating the test signature. In this way, the test signature can be generated with atmospheric leak correction, providing a more accurate measure of the actual leak rate for the test object.

FIG. 1 illustrates an example of an RGA testing system 100 supporting correction for atmospheric leaks in RGA testing in accordance with this disclosure. The embodiment of the testing system 100 shown in FIG. 1 is for illustration only. Other embodiments of the testing system 100 may be used without departing from the scope of this disclosure.

According to embodiments of this disclosure, the testing system 100 can include testing equipment 102 and a testing processor 104, which can be communicatively coupled to each other. The testing system 100 can be located in a testing environment 106, which can include a manufacturing floor, a production floor, a laboratory, or any other suitable type of room, space, or area.

The testing equipment 102 can include a test chamber 108, an RGA 110, and an atmospheric leak standard 112. In addition, the testing equipment 102 can include a plurality of valves 114, such as valves 114a and 114b, for allowing and prohibiting the flow of gases through the testing equipment 102. The testing equipment 102 and the testing processor 104 can be used to provide RGA testing of a test object 116, such as a vacuum-sealed container or the like. For example, in some cases, the test object 116 can include a high-pressure bottle of a specified gas, such as a bottle containing a gas at 8,000 psi or other suitable high pressure.

As described in more detail below, the testing processor 104 can monitor and control the testing equipment 102. For example, among other things, the testing processor 104 can control the pumping down of the test chamber 108 by one or more pumps (not shown in FIG. 1) and can monitor pressure levels for the testing equipment 102 based on measurements provided by one or more pressure gauges (not shown in FIG. 1). In addition, the testing processor 104 can control the valves 114 to open or close to direct the flow of gas within the testing equipment 102.

The test chamber 108 can include an enclosed space that can be pumped down to a near vacuum. When a test object 116 is placed inside the test chamber 108 and a near vacuum has been established within the test chamber 108, the testing processor 104 can signal the valve 114a to open to allow any gases in the test chamber 108 to flow into the RGA 110. The RGA 110 can identify the gases from the test chamber 108 and generate data for determining a flow rate for the gases. In this way, the RGA 110 can be used to measure any leaks in the test object 116 and to verify the leaks are within associated limits for the particular test object 116.

The testing environment 106 for the testing system 100 can include a testing atmosphere 118. The testing atmosphere 118 can include normal atmospheric components, such as nitrogen, oxygen, argon, and carbon dioxide. However, the testing atmosphere 118 can also include ancillary gases 120 emanating from various gas sources 122 within the testing environment 106, such as additional vacuum-sealed containers or any other type of gas source. In addition to these ancillary gases 120, the make-up of the atmosphere 118 in the testing environment 106 can be modified by the presence of one or more people 124, as well as by the functioning of an air conditioning unit (not shown in FIG. 1), weather changes, and the like. Any people 124 present in the testing environment 106 can alter the proportions of oxygen and carbon dioxide in the atmosphere 118 by inhaling components of the atmosphere 118 and producing exhalation products 126 that include a different breakdown of those components. An air conditioning unit for the testing environment 106 and/or changes in the weather can affect temperature, pressure, humidity, and the like for the testing environment 106, which can also change the make-up of the testing atmosphere 118.

Because the test chamber 108 may not be perfectly sealed off from the testing atmosphere 118, atmospheric leaks 128 can introduce components of the testing atmosphere 118 into the test chamber 108. When the RGA 110 is used to measure leaks in the test object 116, these atmospheric leaks 128 can contaminate a chamber signal 130 sent from the test chamber 108 to the RGA 110, making it appear as though they are leaks from the test object 116 instead of atmospheric leaks 128 into the test chamber 108. Although extraneous gases may enter the test chamber 108 in ways other than through atmospheric leaks 128 (such as desorption from the interior wall of the test chamber 108, bulk diffusion, evaporation, or virtual leaks), atmospheric leaks 128 are generally by far the largest source. Thus, by estimating the amount of gases introduced into the test chamber 108 by atmospheric leaks 128 and removing those gases from the chamber signal 130 received during testing, a much more accurate leak rate may be determined for the test object 116.

In operation, after the testing processor 104 directs the establishment of a near vacuum within the test chamber 108 as described in more detail below, the testing processor 104 can signal the valve 114a to close and the valve 114b to open so the atmospheric leak standard 112 can provide a sample 132 of the testing atmosphere 118 to the RGA 110 through a leak signal 134. In some embodiments, the atmospheric leak standard 112 can include a capillary leak circuit that has been calibrated to provide a gas flow at a specified rate at a specified pressure. As a particular example, the atmospheric leak standard 112 can include a capillary leak circuit that has been calibrated to provide a gas flow at 1×10⁻⁷ standard cubic centimeters/second. Thus, when the valve 114b is opened, a sample 132 of the testing atmosphere 118 can flow through the atmospheric leak standard 112, providing a calibrated leak signal 134 to the RGA 110. The RGA 110 can measure the components currently present in the atmosphere 118, as well as their concentrations, based on the leak signal 134.

After the RGA 110 makes this measurement, the testing processor 104 can signal the valve 114b to close and the valve 114a to open in order to allow the RGA 110 to measure the gas flow from the test chamber 108 based on the chamber signal 130. The testing processor 104 can use the measured concentrations of the components in the testing atmosphere 118 provided by the RGA 110 as an estimate of the atmospheric leaks 128 showing up in the chamber signal 130 from the test chamber 108. The testing processor 104 can remove the estimated atmospheric leaks 128 from the chamber signal 130. By measuring the leak signal 134 in close time proximity to the test measurement provided through the chamber signal 130, the leak signal 134 can account for the actual atmospheric conditions at testing, such as pressure, humidity, the actual gas composition of the testing atmosphere 118, and the like.

The measurements of the testing atmosphere 118 through the leak signal 134 and of the test object 116 through the chamber signal 130 can be performed within a specified time period of each other to ensure the measured testing atmosphere 118 accurately reflects the testing atmosphere 118 during testing of the test object 116. For example, in some embodiments, these measurements can be made within 5 minutes, 10 minutes, 15 minutes or any other suitable time period of each other. In this way, the RGA 110 can provide an accurate atmospheric leak estimate based on the leak signal 134 and the testing processor 104 can remove atmospheric leaks 128 from the chamber signal 130 and generate a more accurate measurement of leaks emanating from the test object 116.

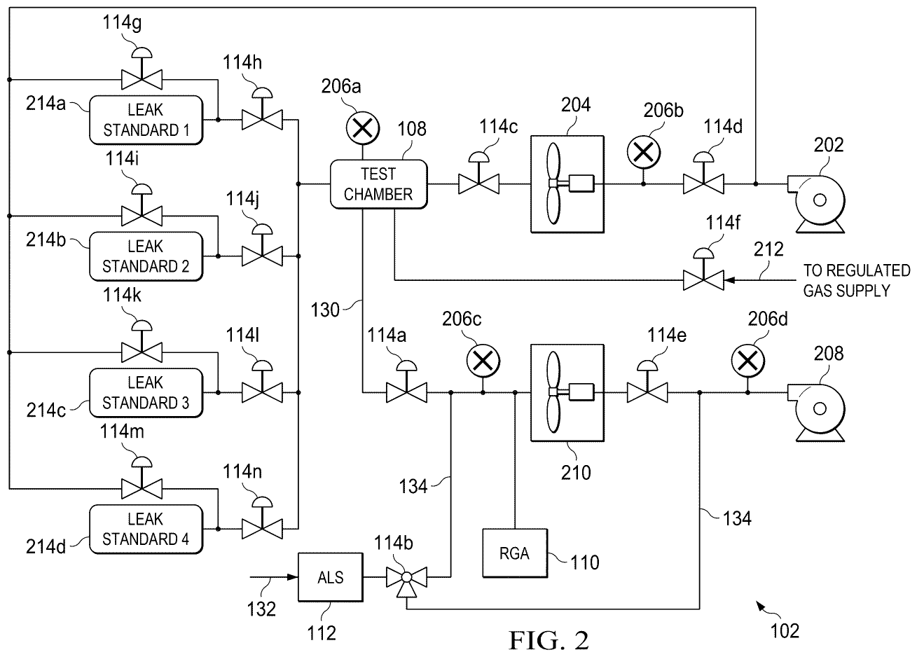

Although FIG. 1 illustrates one example of an RGA testing system 100 supporting correction for atmospheric leaks 128 in RGA testing, various changes may be made to FIG. 1. For instance, although shown and described as leaks into the test chamber 108, the atmospheric leaks 128 can introduce components of the testing atmosphere 118 in other locations within the testing system 100, not just directly into the test chamber 108. For example, atmospheric leaks 128 can exist in pressure gauges, valves, or the like. Also, as described in more detail below in connection with FIG. 2, the valve 114b can include a three-way valve in some embodiments. Further, the testing system 100 and the testing environment 106 can include additional components not shown in FIG. 1. In addition, the testing processor 104 need not be physically present in the testing environment 106 with the testing equipment 102 as shown but instead may be in a separate location. Finally, the view shown in FIG. 1 is not to scale.

FIG. 2 illustrates an example of details of the RGA testing equipment 102 according to this disclosure. The embodiment of the testing equipment 102 shown in FIG. 2 is for illustration only. Other embodiments of the testing equipment 102 may be used without departing from the scope of this disclosure.

According to embodiments of this disclosure, in addition to the components described above in connection with FIG. 1, the testing equipment 102 can include additional valves 114, a roughing pump 202, a chamber turbomolecular pump (TMP) 204, a plurality of pressure gauges 206, an RGA forepump 208, an RGA TMP 210, a regulated gas supply input 212, and a plurality of leak standards 214. In some embodiments, the testing processor 104 (not shown in FIG. 2) can be configured to monitor and control the testing equipment 102 by turning on or off the pumps 202, 204, 208 and 210, opening or closing the valves 114, monitoring the pressure gauges 206, and communicating with the RGA 110.

The roughing pump 202 can include a vacuum pump for initially evacuating the test chamber 108 as a first stage in pumping down the test chamber 108 to a specified crossover point, and the chamber TMP 204 can include a vacuum pump as a second stage for pumping down the test chamber 108 to the crossover point. Similarly, the RGA forepump 208 can include a vacuum pump that serves as a first stage in establishing a flow of gas through the testing equipment 102, and the RGA TMP 210 can include a vacuum pump as a second stage for establishing and maintaining the flow of gas through the testing equipment 102. As described in more detail below, the pumps 202, 204, 208 and 210 can function together to establish and maintain a near vacuum inside the test chamber 108.

Each of the pressure gauges 206a-d can provide a measurement of pressure for a corresponding component of the testing equipment 102. Specifically, in some embodiments, the pressure gauge 206a can provide a measurement of the pressure within the test chamber 108, the pressure gauge 206b can provide a measurement of the pressure generated by the flow of gas as directed by the roughing pump 202 and the chamber TMP 204, the pressure gauge 206c can provide a measurement of the pressure in the flow of gas from either the test chamber 108 or the atmospheric leak standard (ALS) 112 to the RGA 110, and the pressure gauge 206d can provide a measurement of the pressure generated by the flow of gas as directed by the RGA forepump 208.

Each of the leak standards 214a-d can include a calibrated standard for a corresponding gas that could potentially be measured as leaks from a test object 116 in the test chamber 108 during RGA testing. As a particular example, the leak standard 214a can include a nitrogen leak standard. For this example, the leak standard 214a can include a capillary leak coupled to a nitrogen source, a bottle of nitrogen that is calibrated to leak at a specified rate, or any other suitable implementation of a calibrated nitrogen leak. In general, the testing processor 104 can open the valve 114g while keeping the valve 114h closed to allow the gas from the leak standard 214a to flow through the roughing pump 202 and be expelled from the testing system 100. When preparing to test for a leak of nitrogen, however, the testing processor 104 can open the valve 114h while keeping the valve 114g closed to allow the gas from the leak standard 214a to flow into the test chamber 108. By providing a flow of nitrogen gas at a known calibrated rate into the test chamber 108, the chamber signal 130 provided to the RGA 110 can be measured and used as a baseline for testing. When an actual test object 116 including nitrogen is subsequently tested for leaks in the test chamber 108, the testing processor 104 can compare the chamber signal 130 provided to the RGA 110 during testing to the previous chamber signal 130 provided to the RGA 110 based on the leak standard 214a in order to determine a leak rate of nitrogen from the test object 116.

In the embodiment illustrated in FIG. 2, the valve 114b includes a three-way valve. In this embodiment, the testing processor 104 can operate the valve 114b such that the leak signal 134 is provided either to the RGA 110 for measurement or to the RGA forepump 208 for removal from the testing system 100. However, similar to each of the leak standards 214a-d, it will be understood that the atmospheric leak standard 112 can alternatively be coupled to two separate valves 114 instead of to a single three-way valve 114b. As with the leak standards 214a-d, the testing processor 104 can open one of the valves 114 while keeping the other valve 114 closed to provide the leak signal 134 either to the RGA 110 for measurement or to the RGA forepump 208 for removal from the testing system 100.

In operation, the test chamber 108 can be vented to the testing atmosphere 118 before a test object 116 is placed inside the test chamber 108. The valve 114a can be closed, and the valves 114c and 114d can be opened. The valve 114b can be operated to allow the leak signal 134 to flow toward the RGA forepump 208 and prevent the leak signal 134 from flowing to the RGA 110. The RGA forepump 208 can pump out of the testing equipment 102 the leak signal 134 received from the atmospheric leak standard 112 while the roughing pump 202 and the chamber TMP 204 pump out the test chamber 108 until the pressure inside the test chamber 108 reaches the specified crossover point as measured by the pressure gauge 206a. Once the pressure reaches the crossover point, the valves 114a and 114e can be opened while the valves 114c and 114d can be closed. The RGA forepump 208 and the RGA TMP 210 can continue to pump out the test chamber 108 until a lower specified pressure is reached such that the test chamber 108 has a near vacuum established inside.

In some embodiments, the testing processor 104 can ensure proper functionality of the testing system 100 after the test chamber 108 has reached the lower specified pressure and before the actual testing process is performed. For example, the RGA 110 can take a reading based on the chamber signal 130 to verify the testing equipment 102 is functioning properly, and the testing processor 104 can use the reading in an evaluation of the testing system 100.

The valve 114a can be closed while the valve 114b is operated to redirect the leak signal 134 from the RGA forepump 208 to the RGA 110. The sample 132 of the testing atmosphere 118 that is provided through the atmospheric leak standard 112 at a calibrated rate to generate the leak signal 134 is provided to the RGA 110. After a short stabilization period, the RGA 110 takes a measurement of the leak signal 134 and generates a known leak signature based on the leak signal 134. In some embodiments, the short stabilization period can be about one minute.

The valve 114b can be operated to redirect the leak signal 134 back to the RGA forepump 208 and away from the RGA 110 while the valve 114a is opened to allow the chamber signal 130 to be provided to the RGA 110. After a short stabilization period, the RGA 110 takes a measurement of the chamber signal 130 and generates a chamber signature based on the chamber signal 130. In some embodiments, the short stabilization period can be about one minute.

The testing processor 104 can subtract the leak signature from the chamber signature to remove the contamination caused by atmospheric leaks 128 into the test chamber 108 or into other components of the testing system 100, such as valves 114 or pressure gauges 206. In this way, the testing processor 104 can generate a test signature for the test object 116 that has been corrected for atmospheric leaks 128, so the test signature represents a more accurate measure of the actual leak rate for the test object 116.

After the testing process is completed, the valve 114f can be opened to provide an input 212 of a regulated gas supply to the test chamber 108 in order to normalize the pressure and allow the test chamber 108 to be opened for removal of the test object 116. In some embodiments, the regulated gas supply can include a dry nitrogen gas or other suitable dry gas to avoid introducing humidity into the test chamber 108. However, in embodiments in which the test chamber 108 is large enough for a person 124 to enter into the test chamber 108, the testing atmosphere 118 or other suitable atmosphere may be introduced as the input 212 in order to avoid possibly dangerous health effects based on non-standard atmospheric components and/or make-up.

Although FIG. 2 illustrates one example of details of the RGA testing equipment 102, various changes may be made to FIG. 2. For instance, the testing equipment 102 can include any suitable number of leak standards 214. Also, the testing equipment 102 can include additional components not shown in FIG. 2. Further, each pair of valves 114 corresponding to a particular leak standard 214 could be replaced by a three-way valve similar to the valve 114b for the atmospheric leak standard 112. In addition, the view shown in FIG. 2 is not to scale.

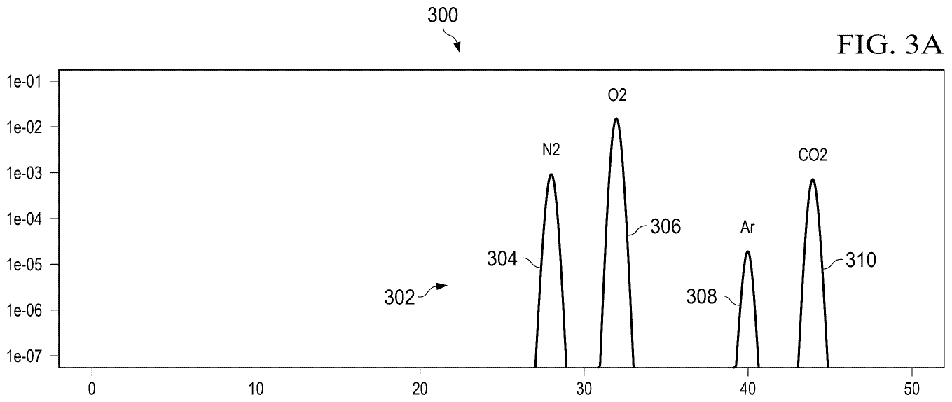

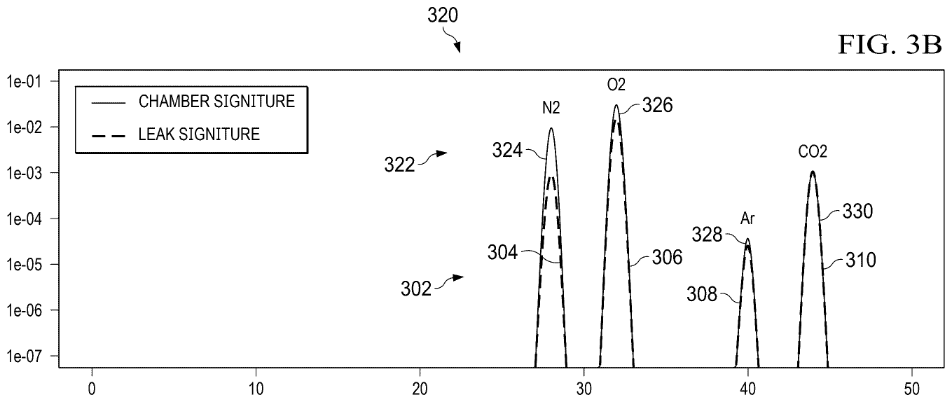

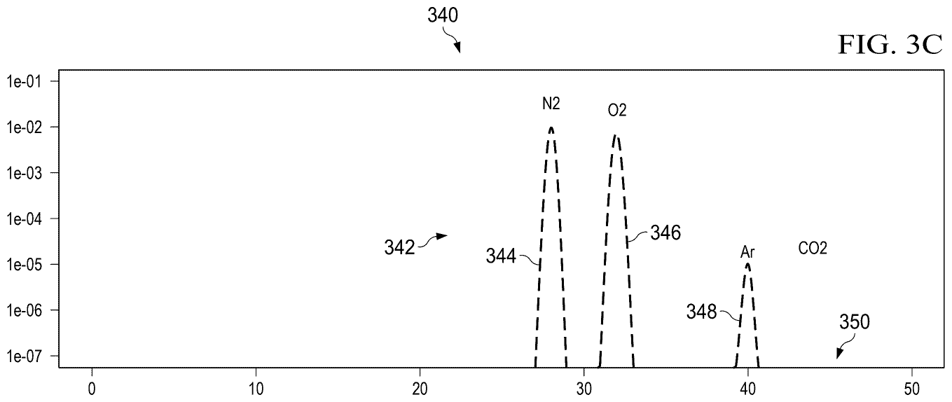

FIGS. 3A through 3C illustrate graphs 300, 320 and 340 depicting examples of RGA spectrums generated during RGA testing with atmospheric leak correction according to this disclosure. Specifically, FIG. 3A illustrates a graph 300 depicting an example of an RGA spectrum corresponding to a known leak signature 302 generated during atmospheric composition estimation for use in atmospheric leak correction according to this disclosure. The graph 300 shown in FIG. 3A is for illustration only. The graph 300 includes an example of a leak signature 302 generated by the RGA 110 based on a leak signal 134. In this embodiment, the sample 132 of the testing atmosphere 118 provided through the leak signal 134 includes various amounts of nitrogen 304, oxygen 306, argon 308, and carbon dioxide 310.

FIG. 3B illustrates a graph 320 depicting an example of an RGA spectrum corresponding to a chamber signature 322 generated during RGA testing in comparison with the leak signature 302 of FIG. 3A for use in atmospheric leak correction according to this disclosure. The graph 320 shown in FIG. 3B is for illustration only. The graph 320 includes an example of a chamber signature 322 generated by the RGA 110 based on a chamber signal 130. The chamber signature 322 is shown by the solid lines, while the leak signature 302 is shown by the dashed lines. In this example, the gas flow received through the chamber signal 130 during RGA testing also includes various amounts of nitrogen 324, oxygen 326, argon 328, and carbon dioxide 330.

FIG. 3C illustrates a graph 340 depicting an example of an RGA spectrum corresponding to a test signature 342 generated during RGA testing using atmospheric leak correction according to this disclosure. The graph 340 shown in FIG. 3C is for illustration only. The graph 340 includes an example of a test signature 342 generated by the testing processor 104 based on the leak signature 302 and the chamber signature 322. The testing processor 104 can generate the test signature 342 by subtracting the leak signature 302 from the chamber signature 322 until at least one of the components reaches a zero value. At that point, there is no difference between the leak signature 302 and the chamber signature 322 with respect to that component (or those components). In this way, the leak signature 302 can be removed from the chamber signature 322 to generate the test signature 342 for the test object 116 with substantially no atmospheric leak contamination. Thus, the test signature 342 includes an atmospheric leak-corrected chamber signature for the test object 116.

As a leak rate for the test object 116 will be zero or positive, the testing processor 104 can ensure that the leak rate is not negative by not subtracting more from the chamber signature 322 than exists in the chamber signature 322. To accomplish this, the testing processor 104 can stop subtracting the leak signature 302 from the chamber signature 322 when at least one of the components reaches a zero value. For example, in a particular case where the leak signature 302 might include a higher value for argon 308 than the value for argon 328 included in the chamber signature 322, the testing processor 104 could subtract the amount of argon 308 from the argon 328 until reaching a zero value for the argon 348 in the test signature 342 so as not to generate a test signature 342 including a negative value for argon 348.

As illustrated in the graphs 320 and 340, the leak signature 302 in this example has been subtracted from the chamber signature 322 until the difference between the leak rate of carbon dioxide 310 in the leak signature 302 and the leak rate of carbon dioxide 330 in the chamber signature 322 reaches zero. However, the differences in the remaining components in the illustrated example are non-zero. Thus, the testing processor 104 can generate the test signature 342 based on the differences between the nitrogen 324 and 304, the oxygen 306 and 326, and the argon 308 and 328.

In the illustrated example, these differences are shown in the amounts of nitrogen 344, oxygen 346, argon 348, and carbon dioxide 350 remaining after the leak signature 302 has been subtracted from the chamber signature 322. Thus, in this example, it can be assumed that some nitrogen 344, oxygen 346, and argon 348 are leaking from the test object 116 at rates corresponding to the values shown in the test signature 342, while no carbon dioxide 350 is leaking from the test object 116, resulting in no carbon dioxide 350 appearing in the test signature 342.

Although FIGS. 3A through 3C illustrate examples of a leak signature 302, a chamber signature 322, and a test signature 342 generated during RGA testing with atmospheric leak correction, various changes may be made to FIGS. 3A through 3C. For instance, in embodiments in which the test object 116 includes a single component that is included in the testing atmosphere 118, the differences in the leak signature 302 and the chamber signature 322 for each of the remaining components could each be substantially zero. For example, if the test object 116 included a bottle of nitrogen, the differences between the oxygen, argon, and carbon dioxide values in the leak signature 302 and the chamber signature 322 could each be substantially zero, and those components would be absent from the test signature 342. However, note that trace amounts of other extraneous gases could appear in the test signature 342, although the amounts of those extraneous gases are generally much lower than amounts corresponding to atmospheric leaks 128.

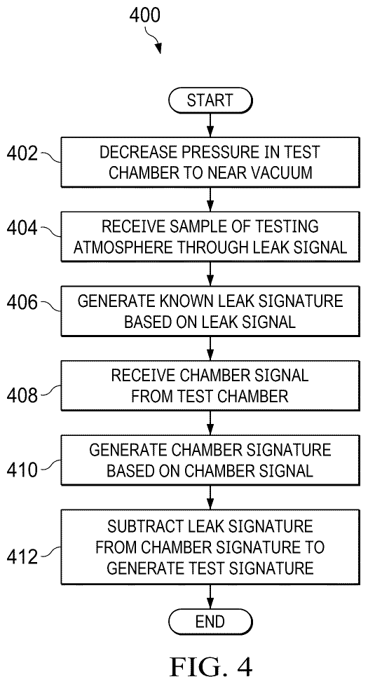

FIG. 4 illustrates an example of a method 400 for correcting for atmospheric leaks 128 in RGA testing according to this disclosure. As shown in FIG. 4, pressure in the test chamber 108 is decreased to a near vacuum at step 402. This may include, for example, the testing processor 104 signaling the valve 114a to close and the valves 114c and 114d to open, as well as prompting the roughing pump 202 and the chamber TMP 204 to pump out the test chamber 108 until the pressure inside the test chamber 108 reaches a specified crossover point. The testing processor 104 can determine whether the crossover point has been reached based on a reading from the pressure gauge 206a. This may also include, in response to the testing processor 104 determining that the pressure has reached the crossover point, the testing processor 104 signaling the valves 114a and 114e to open and the valves 114c and 114d to close and prompting the RGA forepump 208 and the RGA TMP 210 to pump out the test chamber 108 until a lower specified pressure is reached such that the test chamber 108 has a near vacuum established inside. The testing processor 104 can determine whether the pressure in the test chamber 108 has reached a near vacuum based on a reading from the pressure gauge 206a. In some embodiments, while the pressure in the test chamber 108 is being decreased to a near vacuum at step 402, the testing processor 104 can also signal the valve 114b to direct a leak signal 134 toward the RGA forepump 208 to prevent the leak signal 134 from flowing to the RGA 110. While the valve 114b is directing the leak signal 134 toward the RGA forepump 208, the testing processor 104 can also prompt the RGA forepump 208 to pump the leak signal 134 out of the testing system 100.

A sample 132 of the testing atmosphere 118 is received through the leak signal 134 at step 404. This may include, for example, the testing processor 104 signaling the valve 114a to close and signaling the valve 114b to direct the leak signal 134 to the RGA 110. In this way, the RGA 110 can receive the leak signal 134 that has been generated based on a sample 132 of the testing atmosphere 118 passing through the atmospheric leak standard 112 at a calibrated rate. A known leak signature 302 is generated based on the leak signal 134 at step 406. This may include, for example, the RGA 110 generating an RGA spectrum, such as illustrated in FIG. 3A, corresponding to the leak signal 134. Thus, the leak signature 302 can include information related to leak rates of various atmospheric components that could be present in atmospheric leaks 128 into the test chamber 108 or other components of the testing equipment 102. In some embodiments, the testing processor 104 can prompt the RGA 110 to generate the leak signature 302 after a specified stabilization period for the leak signal 134. As a particular example, the specified stabilization period can be about one minute after the RGA 110 begins receiving the leak signal 134.

A chamber signal 130 is received from the test chamber 108 at step 408. This may include, for example, the testing processor 104 signaling the valve 114b to direct the leak signal 134 back to the RGA forepump 208 and away from the RGA 110 and signaling the valve 114a to open, thereby allowing the chamber signal 130 to be provided from the test chamber 108 to the RGA 110. In addition, while the valve 114b is directing the leak signal 134 toward the RGA forepump 208, the testing processor 104 can again prompt the RGA forepump 208 to pump the leak signal 134 out of the testing system 100. In some embodiments, the chamber signal 130 may be received from the test chamber 108 within a specified time period of receiving the leak signal 134 at step 404 to ensure the leak signal 134 accurately reflects the testing atmosphere 118 while the chamber signal 130 is received. For example, in some embodiments, these signals 130 and 134 can be received within 5 minutes, 10 minutes, 15 minutes or any other suitable time period of each other.

A chamber signature 322 is generated based on the chamber signal 130 at step 410. This may include, for example, the RGA 110 generating an RGA spectrum, such as illustrated in FIG. 3B, corresponding to the chamber signal 130. Thus, in addition to information related to one or more gases that may be leaking from the test object 116, the chamber signature 322 can also include information related to various atmospheric components that could be present in atmospheric leaks 128 into the test chamber 108 or other components of the testing equipment 102. In some embodiments, the testing processor 104 can prompt the RGA 110 to generate the chamber signature 322 after a specified stabilization period for the chamber signal 130. As a particular example, the specified stabilization period can be about one minute after the RGA 110 begins receiving the chamber signal 130. The stabilization period before generating the chamber signature 322 can be the same as or different from the stabilization period before generating the leak signature 302.

The leak signature 302 is subtracted from the chamber signature 322 to generate a test signature 342 at step 412. This may include, for example, the testing processor 104 generating the test signature 342 by subtracting the leak signature 302 from the chamber signature 322 to remove contamination caused by atmospheric leaks 128 into the test chamber 108 or elsewhere in the testing equipment 102 until at least one of the components reaches a zero value. In this way, the testing processor 104 can correct the chamber signature 322 for atmospheric leaks 128 based on the leak signature 302 to generate the test signature 342 for the test object 116, and the test signature 342 is a more accurate measure of the actual leak rate for the test object 116.

Although FIG. 4 illustrates one example of a method 400 for correcting for atmospheric leaks 128 in RGA testing, various changes may be made to FIG. 4. For example, in some embodiments, the testing processor 104 can ensure proper functionality of the testing system 100 after the pressure in the test chamber 108 has reached a near vacuum at step 402 before receiving the sample 132 of the testing atmosphere 118 at step 404. In particular embodiments, the testing processor 104 can prompt the RGA 110 to take a reading based on the chamber signal 130 to verify the testing equipment 102 is functioning properly, and the testing processor 104 can use the reading (in addition to any other suitable criteria) in an evaluation of the testing system 100. Also, while shown as a series of steps, various steps in FIG. 4 may overlap, occur in parallel, occur in a different order, or occur any number of times (including zero times). For example, in some embodiments, the chamber signal 130 may be received and the chamber signature 322 may be generated before the leak signal 134 is received and the leak signature 134 is generated.

In some embodiments, various functions described in this patent document are implemented or supported by a computer program or other program that is formed from computer readable program code or instructions and that is embodied in a computer or machine readable medium. The phrases “computer readable program code” and “instructions” include any type of code, including source code, object code, and executable code. The phrases “computer readable medium” and “machine readable medium” include any type of medium capable of being accessed by a computer or other machine, such as read only memory (ROM), random access memory (RAM), a hard disk drive (HDD), a compact disc (CD), a digital video disc (DVD), or any other type of memory. A “non-transitory” computer or machine readable medium excludes wired, wireless, optical, or other communication links that transport transitory electrical or other signals. A non-transitory computer or machine readable medium includes media where data can be permanently stored and media where data can be stored and later overwritten, such as a rewritable optical disc or an erasable storage device.

It may be advantageous to set forth definitions of certain words and phrases used throughout this patent document. The term “about” (when used with a numerical value) indicates that the numerical value may vary by up to ±10%. The terms “application” and “program” refer to one or more computer programs, software components, sets of instructions, procedures, functions, objects, classes, instances, related data, or a portion thereof adapted for implementation in a suitable computer code (including source code, object code, or executable code). The term “communicate,” as well as derivatives thereof, encompasses both direct and indirect communication. The terms “include” and “comprise,” as well as derivatives thereof, mean inclusion without limitation. The term “or” is inclusive, meaning and/or. The phrase “associated with,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, have a relationship to or with, or the like. The phrase “at least one of,” when used with a list of items, means that different combinations of one or more of the listed items may be used, and only one item in the list may be needed. For example, “at least one of: A, B, and C” includes any of the following combinations: A, B, C, A and B, A and C, B and C, and A and B and C.

The description in the present application should not be read as implying that any particular element, step, or function is an essential or critical element that must be included in the claim scope. The scope of patented subject matter is defined only by the allowed claims. Moreover, none of the claims invokes 35 U.S.C. § 112(f) with respect to any of the appended claims or claim elements unless the exact words “means for” or “step for” are explicitly used in the particular claim, followed by a participle phrase identifying a function. Use of terms such as (but not limited to) “mechanism,” “module,” “device,” “unit,” “component,” “element,” “member,” “apparatus,” “machine,” “system,” “processor,” or “controller” within a claim is understood and intended to refer to structures known to those skilled in the relevant art, as further modified or enhanced by the features of the claims themselves, and is not intended to invoke 35 U.S.C. § 112(f).

While this disclosure has described certain embodiments and generally associated methods, alterations and permutations of these embodiments and methods will be apparent to those skilled in the art. Accordingly, the above description of example embodiments does not define or constrain this disclosure. Other changes, substitutions, and alterations are also possible without departing from the spirit and scope of this disclosure, as defined by the following claims.

What is claimed is:

1. A method comprising:
generating a known leak signature based on a leak signal received at a residual gas analyzer (RGA);
generating a chamber signature based on a chamber signal received at the RGA from a test chamber while a test object is inside the test chamber; and
correcting the chamber signature for atmospheric leaks based on the leak signature to generate a test signature, wherein the test signature corresponds to an atmospheric leak-corrected chamber signature for the test object.

2. The method of claim 1, wherein correcting the chamber signature for atmospheric leaks based on the leak signature to generate the test signature comprises subtracting the leak signature from the chamber signature.

3. The method of claim 1, wherein the leak signal is generated by an atmospheric leak standard based on a sample of a testing atmosphere.

4. The method of claim 3, further comprising:
receiving the leak signal from the atmospheric leak standard at the RGA;
wherein generating the leak signature comprises generating the leak signature after receiving the leak signal for at least an amount of time corresponding to a stabilization period for the leak signal.

5. The method of claim 4, wherein the stabilization period for the leak signal is about one minute.

6. The method of claim 3, wherein the atmospheric leak standard comprises a capillary leak circuit.

7. The method of claim 3, wherein:
the testing atmosphere comprises a plurality of atmospheric components;
the leak signature, the chamber signature, and the test signature each comprise at least a subset of the atmospheric components; and
correcting the chamber signature for atmospheric leaks based on the leak signature to generate the test signature comprises subtracting the leak signature from the chamber signature until at least one of the atmospheric components in the test signature reaches a value of zero.

8. The method of claim 1, further comprising:
receiving the chamber signal at the RGA;
wherein generating the chamber signature comprises generating the chamber signature after receiving the chamber signal for at least an amount of time corresponding to a stabilization period for the chamber signal.

9. The method of claim 8, wherein the stabilization period for the chamber signal is about one minute.

10. A method comprising:
receiving a sample of a testing atmosphere at a residual gas analyzer (RGA) through a leak signal;
generating a known leak signature based on the leak signal;
receiving a chamber signal at the RGA from a test chamber while a test object is inside the test chamber;
generating a chamber signature based on the chamber signal; and
correcting the chamber signature for atmospheric leaks based on the leak signature to generate a test signature, wherein the test signature corresponds to an atmospheric leak-corrected chamber signature for the test object.

11. The method of claim 10, wherein correcting the chamber signature for atmospheric leaks based on the leak signature to generate the test signature comprises subtracting the leak signature from the chamber signature.

12. The method of claim 10, further comprising:
generating the leak signal based on the sample of the testing atmosphere with an atmospheric leak standard;
wherein receiving the sample of the testing atmosphere at the RGA through the leak signal comprises receiving the leak signal at the RGA from the atmospheric leak standard.

13. The method of claim 12, wherein generating the leak signature comprises generating the leak signature after receiving the leak signal at the RGA from the atmospheric leak standard for at least an amount of time corresponding to a stabilization period for the leak signal.

14. The method of claim 12, wherein the atmospheric leak standard comprises a capillary leak circuit.

15. The method of claim 10, wherein:
the testing atmosphere comprises a plurality of atmospheric components;
the leak signature, the chamber signature, and the test signature each comprise at least a subset of the atmospheric components; and
correcting the chamber signature for atmospheric leaks based on the leak signature to generate the test signature comprises subtracting the leak signature from the chamber signature until at least one of the atmospheric components in the test signature reaches a value of zero.

16. The method of claim 10, wherein generating the chamber signature comprises generating the chamber signature after receiving the chamber signal at the RGA from the test chamber for at least an amount of time corresponding to a stabilization period for the chamber signal.

17. A system comprising:
a test chamber configured to generate a chamber signal while a test object is inside the test chamber;
at atmospheric leak standard configured to receive a sample of a testing atmosphere and to generate a leak signal based on the sample;
a residual gas analyzer (RGA) coupled to the test chamber and the atmospheric leak standard, wherein the RGA is configured to receive the leak signal and generate a known leak signature based on the leak signal and to receive the chamber signal and generate a chamber signature based on the chamber signal; and
a testing processor communicatively coupled to the RGA, wherein the testing processor is configured to correct the chamber signature for atmospheric leaks based on the leak signature to generate a test signature, and wherein the test signature corresponds to an atmospheric leak-corrected chamber signature for the test object.

18. The system of claim 17, wherein the testing processor is configured to correct the chamber signature for atmospheric leaks based on the leak signature to generate the test signature by subtracting the leak signature from the chamber signature.

19. The system of claim 18, wherein:
the testing atmosphere comprises a plurality of atmospheric components;
the leak signature, the chamber signature, and the test signature each comprise at least a subset of the atmospheric components; and
to subtract the leak signature from the chamber signature, the testing processor is configured to subtract the leak signature from the chamber signature until at least one of the atmospheric components in the test signature reaches a value of zero.

20. The system of claim 17, wherein the atmospheric leak standard comprises a capillary leak circuit.
